(19) United States
(12) Patent Application Publication
(10) Pub. No.: US 2006/0040259 A1
(43) Pub. Date: Feb. 23, 2006

(54) Method for inhibition of viral infection

(76) Inventor: Jeffrey S. Glenn, Palo Alto, CA (US)

Correspondence Address: MORRISON & FOERSTER LLP, 12531 HIGH BLUFF DRIVE, SUITE 100, SAN DIEGO, CA, 92130-2040, US

(21) Appl. No.: 11/000,691

(22) Filed: Nov. 30, 2004

Related U.S. Application Data
(63) Continuation of application No. 10/317,644, filed on Dec. 11, 2002, now abandoned.
Application No. 10/317,644 is a continuation-in-part of application No. 09/687,267, filed on Oct. 13, 2000, now Pat. No. 6,627,610.
Application No. 09/687,267 is a division of application No. 09/028,655, filed on Feb. 24, 1998, now Pat. No. 6,159,939.
Application No. 09/028,655 is a continuation of application No. 08/347,448, filed on Jun. 23, 1995, now Pat. No. 5,876,920.
Application No. 08/347,448 is a continuation-in-part of application No. 07/890,754, filed on May 29, 1992, now Pat. No. 5,503,973.

(51) Int. Cl. C12Q 1/70; A61K 38/06

(52) U.S. Cl. 435/5; 514/18

(57) ABSTRACT

The invention is directed to inhibiting viral morphogenesis and viral infection. In particular, it concerns effecting such inhibition by inhibiting the prenylation or post prenylation reactions of a viral or host protein.

### CROSS-REFERENCE TO RELATED APPLICATIONS

This application is continuation-in-part of U.S. Ser. No. 09/687,267, filed Oct. 13, 2000, now allowed, which is a divisional of U.S. Ser. No. 09/028,655, filed Feb. 24, 1998, now U.S. Pat. No. 6,159,939, which is a continuation of U.S. Ser. No. 08/347,448, filed Jun. 23, 1995, now U.S. Pat. No. 5,876,920, which is a 371 of PCT/US93/05247, filed Jun. 1, 1993, which is a Continuation-in-Part of U.S. Ser. No. 07/890,754, filed May 29, 1992, now U.S. Pat. No. 5,503,973. The disclosures of the above-referenced applications are incorporated in their entirety by reference.

### STATEMENT REGARDING FEDERALLY SPONSORED RESEARCH OR DEVELOPMENT

This invention was partially supported by the Medical Scientist Training Program and Veteran Administration Merit Review Award. The government has certain rights to this invention.

### BACKGROUND OF THE INVENTION

The invention is directed to inhibiting viral morphogenesis and viral infection. In particular, it concerns effecting such inhibition by inhibiting the prenylation or post prenylation reactions of a viral or host protein.

It has been shown that certain membrane-associated proteins require the addition of lipophilic residues in order to function properly. One family of such modifications is termed “prenylation” because the hydrophobic residue is derived from isoprenoid precursors. The prenyl residue is known to attach to the sulfhydryl group of a cysteine which has been shown in a number of membrane-associated proteins to be contained in a “CXXX” (SEQ ID NO:1) box at the carboxy terminus of the substrate protein, wherein C means cysteine (Cys) and X means any amino acid residue. In particular, one such membrane-associated protein has been shown to be the protein product of the ras oncogene. Summaries of these reactions conferring hydrophobic properties on membrane proteins, including prenylation, have appeared by Hoffman, M., Science (1991) 254:650-651, and by Gibbs, J. B., Cell (1991) 65:1-4.

In addition, in many cases, prenylation is a first step in a series of further reactions which modify the carboxy terminus of prenylated proteins. These prenylation initiated, or post-prenylation reactions include proteolysis and carboxymethylation.

In many of the prenylation substrate proteins studied to date, the CXXX (SEQ ID NO:1) box contains aliphatic residues in the second and third positions and a leucine, serine, methionine, cysteine or alanine in the terminal position. Thus, in the CXXX boxes so far studied, the box itself is relatively hydrophobic.

It has now been found that prenylation of a viral protein is necessary for the morphogenesis of hepatitis delta virus (HDV). This is the first demonstration that viral proteins are subject to prenylation. Furthermore, certain functional consequences can be ascribed to prenylation. The viral protein which is the target of prenylation, surprisingly, contains a hydrophilic CXXX (SEQ ID NO:1) box of the sequence Cys-Arg-Pro-Gln (SEQ ID NO:2). Prenylation, or prenylation-initiated modification, of this relatively hydrophilic CXXX box and corresponding CXXX (SEQ ID NO:1) boxes (hydrophilic or otherwise) or other cysteine-containing sequences near the C-terminus of proteins in other virions are suitable targets for antiviral strategies.

These targets can now be seen to include, but are not limited to, proteins of hepatitis A virus (HAV), hepatitis C virus (HCV), herpes simplex virus (HSV), cytomegalovirus (CMV), varicella-zoster virus (VZV), influenza virus, plant viruses such as tobacco mosaic satellite virus (TMSV) and barley stripe mosaic virus (BSMV), the core antigen of hepatitis B virus (HBV) and the nef gene product of human immunodeficiency virus-1 (HIV-1)—especially since nef has been shown to play an important role in the development of AIDS. (Kesstler, H. W. III, et al. Cell (1991) 65:651-662. Accordingly, inhibition of the prenylation of these target proteins or the post-prenylation reactions thereof is claimed to be inhibitory to the progress of these infections.

### BRIEF SUMMARY OF THE INVENTION

The invention provides methods to interfere with viral morphogenesis, production, release or uncoating both in vitro and in vivo. Agents which interfere with the prenylation of, or the post-prenylation reactions of, at least one viral protein are provided to infected cells to halt the viral infection. Such cells may be in culture or may be contained in an animal or plant subject.

Thus, in one aspect, the invention is directed to a method to inhibit viral morphogenesis, production, release or uncoating which method comprises effectively interfering with the prenylation of, or the post-prenylation reactions of, at least one viral protein.

In another aspect, the invention is directed to an assay method for screening candidate drugs for their ability to inhibit prenylation. In a third aspect, the invention is directed to a method for treating viral infection by administering an agent effective to inhibit prenylation of, or the post-prenylation reactions of, a viral protein. In preferred embodiments, the viral protein is the large delta antigen of the hepatitis D virus or 3 D protein of hepatitis A virus.

In still another aspect, the invention is directed to a method to treat a viral infection in a subject via inhibiting the prenylation or a post-prenylation reaction of a protein contained in the virus infecting said subject, which method comprises administering to said subject an effective amount of an agent selected from the group consisting of a peptide that mimics the amino acid sequence of a “CXXX” (SEQ ID NO:1), “XCXX” (SEQ ID NO:3), “XXCX” (SEQ ID NO:4), or “XXXC” (SEQ ID NO:5) box as it occurs in said viral protein, an inhibitor of a prenyl transferase, an inhibitor of an enzyme included in the pathway of a prenyl lipid synthesis from mevalonate, a mimic of a prenyl group, an inhibitor of a protease that removes the XXX tripeptide from the CXXX polypeptide following prenylation, a protease that removes the XX dipeptide from the XCXX polypeptide following prenylation, or a protease that removes the X residue from the XXCX polypeptide following prenylation, or a protease that removes a C-terminal domain of the prenylated protein including the entire CXXX box, an inhibitor of prenyl cysteine methyltransferase, and a combination thereof. Exemplary combination includes a combination of lovastatin, an inhibitor of an enzyme included in the pathway of a prenyl lipid synthesis from mevalonate, and 3-allylfarnesol, an inhibitor of protein farnesyltransferase (Mattingly et al., J. Pharmacol. Exp. Ther., 303(l):74-81 (2002)). Preferably, the agent is administered with a pharmaceutically acceptable carrier or excipient.

In a specific embodiment, the agent is an inhibitor of an enzyme along the pathway of prenyl lipid synthesis from mevalonate i.e., one of the enzymes involved in the biosynthetic pathway which starts from HMG-CoA and ends with a fully-formed prenyl group ready to be transferred to a target protein, proceeding through mevalonate. Such exemplary enzymes include HMG-CoA-reductase (Lutz et al., Proc. Natl. Acad. Sci. USA, 89(7 :3000-4 (1992); Erratum in: Proc. Natl. Acad. Sci. USA, 89(12):5699 (1992)) and farnesyl diphosphate synthase (Dunford et al., J. Pharmacol. Exp. Ther., 296(2):235-42 (2001)).

In another specific embodiment, the agent is a mimic of a prenyl group such as beta-ketophosphonic acid alone, or with fluorines incorporated at the alpha position (Kang et al., Biochem. Biophys. Res. Commun., 217(l):245-9 (1995)). “A mimic of a prenyl group” should behave like a prenyl group, e.g., farnesyl diphosphate, but cannot be used as a prenyl group donor in a functional prenylation reaction. In one aspect, “a mimic of a prenyl group” can behave as a competitive inhibitor of a prenyl group donor in a prenylation reaction. Such a competitive inhibitor is disclosed in Pompliano et al., Biochemistry, 31:3800-3807 (1992). Pompliano et al. showed that two nonhydrolyzable analogues of farnesyl diphosphate, (alpha-hydroxyfarnesyl)phosphonic acid (1) and [[(farnesylmethyl)hydroxyphosphinyl]methyl]phosphonic acid (2), are competitive inhibitors of farnesyl diphosphate and noncompetitive inhibitors of Ras-CVLS (SEQ ID NO:6).

However, it should be noted that the above description of a mimic of a prenyl group behaving as a competitive inhibitor in a prenylation reaction is for illustration only. The meaning of the mimic of a prenyl group should not be limited to such competitive inhibitor because the mimic may block the normal prenylation through other mechanism(s). For example, a prenyl group may be modified so that, although it can be used as a prenyl group donor to be transferred to a CXXX box, the modification interferes with the function of the prenyl group, e.g., blocking binding of the modified prenyl group with its receptor. In this way, the modified prenyl group can be used a mimic of the prenyl group because the modified prenyl group blocks functional prenylation of a viral protein with the CXXX box.

In addition, other examples of prenyl group mimics are well known in the art. Such exemplary prenyl group mimics include oreganic acid (Silverman et al., Biochem. Biophys. Res. Commun., 232(2):478-81 (1997)), 2-diazo-3,3,3-trifluoropropionyloxy-farnesyl diphosphate (DATFP-FPP) (Bukhtiyarov et al., J Biol. Chem., 270(32):19035-40 (1995)), 1-phosphono-(E,E,E)-geranylgeraniol, a dead-end inhibitor for GGPP (Stirtan and Poulter, Biochemistry, 36(15):4552-7 (1997)), Cbz-His-Tyr-Ser(OBn)TrpNH2 and Cbz-HisTyr(OP042-)-Ser(OBn)TrpNH2 (Scholten et al., J. Biol. Chem., 272(29):18077-81 (1997)) and alpha-cyanocinnamide derivatives (Poradosu et al., Bioorg. Med. Chem., 7(8):1727-36 (1999)). It is noteworthy that these prenyl group mimics are molecules with distinct structures. Therefore, the term “prenyl group mimics” means, to those skilled in the art, not just a single group, but a diverse group of molecules.

In still another specific embodiment, the agent is an inhibitor of a protease that removes the XXX tripeptide from the CXXX polypeptide following prenylation, a protease that removes the XX dipeptide from the XCXX polypeptide following prenylation, a protease that removes the X residue from the XXCX polypeptide following prenylation, or a protease that removes a C-terminal domain of the prenylated protein including the entire CXXX box. Any suitable proteolytic cleavage inhibitors can be used in the present methods. For example, any such inhibitors disclosed in U.S. Pat. No. 6,391,574 and any such inhibitors obtained by a screening assay disclosed in U.S. Pat. No. 6,391,574 can be used in the present methods.

In yet another specific embodiment, the agent is an inhibitor of prenyl cysteine methyltransferase. Any suitable inhibitors of prenyl cysteine methyltransferase can be used in the present methods. For example, any such inhibitors disclosed in U.S. Pat. Nos. 5,043,268, 6,184,016, 6,232,108 and 6,432,403 and any such inhibitors obtained by a screening assay disclosed in U.S. Pat. Nos. 5,043,268, 6,184,016, 6,232,108 and 6,432,403 can be used in the present methods.

The present methods can be used to treat a viral infection in any suitable subject. Exemplary subjects include animal, plant, fungus and bacterium subjects. In a specific embodiment, the subject to be treated is an animal or a plant. Preferably, the animal is a mammal, e.g., a human or a non-human primate.

The present methods can be used to treat a viral infection caused by any virus whose morphogenesis depends, at least partially, on prenylation of a viral protein or a host protein. For example, the present methods can be used to treat a viral infection that is caused by a double-strand DNA virus, a negative single-strand RNA virus, a positive single-strand RNA virus or a double-strand RNA virus. Exemplary double-strand DNA viruses include a poxviridae, e.g., the orthopoxviruses (of which vaccinia virus and smallpox virus are members), and the molluscipoxviruses, a herpesviridae, e.g., herpes simplex virus (HSV) and varicella zoster virus (VZV), and a papillomaviridiae, e.g., human papilloma virus. Exemplary negative single-strand RNA viruses include a bunyaviridiae, e.g., bunyavirus and oropouche virus. Exemplary positive single-strand RNA viruses include a hepatovirus, e.g., HAV. Exemplary double-strand RNA viruses include a reoviridiae, e.g., the reoviridiae of which reovirus is a member. In a specific embodiment, the present methods can be used to treat a viral infection caused by a pox virus, e.g., smallpox virus, a bunyavirus, e.g., oropouche virus, hepatitis E virus, human papilloma virus, molluscum contagiosum virus, vaccinia virus or reovirus.

In yet another aspect, the invention is directed to a kit to treat a viral infection in a subject via inhibiting the prenylation or a post-prenylation reaction of a protein contained in the virus infecting said subject, which kit comprises in the same container or different containers: a) an effective amount of an agent selected from the group consisting of a peptide that mimics the amino acid sequence of a “CXXX” (SEQ ID NO:1), “XCXX” (SEQ ID NO:3), “XXCX” (SEQ ID NO:4), or “XXXC” (SEQ ID NO: 5) box as it occurs in said viral protein, an inhibitor of a prenyl transferase, an inhibitor of an enzyme included in the pathway of a prenyl lipid synthesis from mevalonate, a mimic of a prenyl group, an inhibitor of a protease that removes the XXX tripeptide from the CXXX polypeptide following prenylation, a protease that removes the XX dipeptide from the XCXX polypeptide following prenylation, or a protease that removes the X residue from the XXCX polypeptide following prenylation, and an inhibitor of prenyl cysteine methyltransferase; and b) an instruction for using said agent in treating said viral infection in said subject.

In yet another aspect, the invention is directed to a method to treat a viral infection in a subject via inhibiting the prenylation or a post-prenylation reaction of a host protein involved in life cycle of said infecting virus, which method comprises administering to said subject an effective amount of an agent selected from the group consisting of a peptide that mimics the amino acid sequence of a “CXXX” (SEQ ID NO:1), “XCXX” (SEQ ID NO:3), “XXCX” (SEQ ID NO:4), or “XXXC” (SEQ ID NO: 5) box as it occurs in said viral protein, an inhibitor of a prenyl transferase, an inhibitor of an enzyme included in the pathway of a prenyl lipid synthesis from mevalonate, a mimic of a prenyl group, an inhibitor of a protease that removes the XXX tripeptide from the CXXX polypeptide following prenylation, a protease that removes the XX dipeptide from the XCXX polypeptide following prenylation, or a protease that removes the X residue from the XXCX polypeptide following prenylation, and an inhibitor of prenyl cysteine methyltransferase. Exemplary viral life cycle events include viral morphogenesis (which may include formation or assembly of the virus particle, etc.), production (which may include production of new viral genomes, genome intermediates, viral gene transcripts or products, or completed virions, etc.), release (which may include entrance into the secretory pathway for exit from the cell, access to the extracellular environment, etc.) or uncoating (which may include removal of virus components upon entrance into a new host cell, or disassembly of virus components upon infection of a new host cell, etc.).

### BRIEF DESCRIPTION OF THE SEVERAL VIEWS OF THE DRAWING(S)

FIGS. 1A and 1B are photocopies of immunoblots of proteins obtained by lysis of viral-infected cells expressing viral proteins and treated with tritiated mevalonate.

FIGS. 2A and 2B are photocopies of immunoblots of proteins derived from lysates of cells containing wild type or mutant viral proteins and labeled with tritiated proline or mevalonate.

FIGS. 3A, 3B, 3C and 3D are photocopies of immunoblots of various cell supernatants containing viral proteins.

FIG. 4 is a diagrammatic representation of the progress of HDV morphogenesis.

FIG. 5 illustrates that CXXX boxes from a variety of viruses are subject to prenylation. CXXX boxes located in the proteins of several different types of viruses were selected and used to replace the normal CXXX box of HDV'S large delta antigen. The resulting chimeric proteins were then synthesized and tested for their ability to undergo prenylation in a rabbit reticulocyte lysate system, as previouly described in Glenn et al., Science, 256:1331-1333 (1992). Large delta antigen (which has a CXXX box capable of undergoing prenylation) and small delta antigen (which does not have a CXXX box capable of undergoing prenylation) served as positive and negative controls, respectively. Note CXXX boxes found in hepatitis A virus (HAV), cytomegalovirus (CMV) and herpes simplex virus (HSV) all undergo prenylation.

FIG. 6 illustrates in vitro production of HDV genome-containing virions. Huh-7 cells were transfected with plasmids encoding the HDV genome and the HBV genome, or with either plasmid alone. Media supernatants collected on the indicated days after transfection were then analyzed by northern blot with a probe for HDV genomic RNA to detect the presence of genome-containing virions. In vitro transcribed linear HDV RNAs (a small fraction of which have undergone autocatalytic processing at the genomic strand ribozyme site) were included on the right side of the blot as standards. Note that the HDV RNA in the virions migrates slightly faster than the linear standards, as is characteristic for circularized genomic RNA contained in intact virions.

FIG. 7 illustrates infection of human primary heptocytes with HDV. Primary human hepatocytes were inoculated with produced HDV particles, cultured for one week, fixed, and stained with a human anti-delta antigen serum as primary antibody and rhodamine-labelled goat anti-human reagent as secondary antibody. Note characteristic nuclear staining pattern of delta antigen in several hepatocytes.

FIG. 8 illustrates that FT1-277 inhibits production of HDV virions. Huh-7 cells were co-transfected with HDV and HBV encoding plasmids to establish production of HDV virions, as described in the text, and grown in the presence of the indicated concentrations of FTI-277. HDV genome replication in the cells, and the amount of HDV genome-containing virions released into the supernatants, were monitored by northern blot analysis with a probe for HDV genomic RNA (left panel). The results were quantitated with a phosphoimager and the amount of virions produced at each concentration of FTI-277, expressed as a percentage of the no drug control, was plotted (purple bars, right panel). Also plotted are the results of assays for cellular metabolism (XTT assay, yellow bars), and general protein synthesis and secretion (HBV surface antigen released into the media supernatants, blue bars).

FIGS. 9A-D illustrate in vivo treatment of hepatitis delta virus (HDV) with the prenylation inhibitors FTI-277 and FTI-2153. HBV-transgenic mice were inoculated by hydrodynamic transfection to initiate authentic HDV genome replication. Mice were treated for one week by IP injection with vehicle alone (lanes 1 and 6), vehicle+50 mg/kg/day FTI-277 (lanes 2-5), or vehicle+50 mg/kg/day FTI-2153 (lanes 7-10). Serum samples were then analyzed for HDV virions by RT-PCR analysis, and non-specific toxicity by ALT assays. The primers used in the RT-PCR assay yield a 540 bp fragment only in the presence of circular viral genomic RNA, as found in virions. Note that the production and release of HDV virions into the serum was completely eliminated in the groups treated with prenylation inhibitors.

FIG. 10 illustrates CXXX box-containing proteins in vaccinia virus and hepatitis A virus.

FIGS. 11 and 12 illustrate the dramatic effect of prenylation inhibitors on vaccinia virus production.

FIG. 13 illustrates a simplified pathway of a prenyl lipid synthesis from mevalonate (Dunford et al., J. Pharmacol. Exp. Ther., 296(2):235-42 (2001)). The reactions indicated are catalyzed by IPP isomerase (1), FPP synthase (2), GGPP synthase (3), protein:farnesyl transferase (4), protein:geranylgeranyl transferase 1 (5) and squalene synthase (6).

### DETAILED DESCRIPTION OF THE INVENTION

For clarity of disclosure, and not by way of limitation, the detailed description of the invention is divided into the subsections that follow.

### A. DEFINITIONS

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “plant” refers to any of various photosynthetic, eucaryotic multi-cellular organisms of the kingdom Plantae, characteristically producing embryos, containing chloroplasts, having cellulose cell walls and lacking locomotion.

As used herein, “animal” refers to a multi-cellular organism of the kingdom of Animalia, characterized by a capacity for locomotion, nonphotosynthetic metabolism, pronounced response to stimuli, restricted growth and fixed bodily structure. Non-limiting examples of animals include birds such as chickens, vertebrates such fish and mammals such as mice, rats, rabbits, cats, dogs, pigs, cows, ox, sheep, goats, horses, monkeys and other non-human primates.

As used herein, “infection” refers to invasion of the body of a multi-cellular organism with organisms that have the potential to cause disease.

As used herein, “infectious organism” refers to an organism that is capable to cause infection of a multi-cellular organism. Most infectious organisms are microorganisms such as viruses, bacteria and fungi.

As used herein, “bacteria” refers to small prokaryotic organisms (linear dimensions of around 1 μm) with non-compartmentalized circular DNA and ribosomes of about 70S. Bacteria protein synthesis differs from that of eukaryotes. Many anti-bacterial antibiotics interfere with bacteria proteins synthesis but do not affect the infected host.

As used herein, “eubacteria” refers to a major subdivision of the bacteria except the archaebacteria. Most Gram-positive bacteria, cyanobacteria, mycoplasmas, enterobacteria, pseudomonas and chloroplasts are eubacteria. The cytoplasmic membrane of eubacteria contains ester-linked lipids; there is peptidoglycan in the cell wall (if present); and no introns have been discovered in eubacteria.

As used herein, “archaebacteria” refers to a major subdivision of the bacteria except the eubacteria. There are 3 main orders of archaebacteria: extreme halophiles, methanogens and sulphur-dependent extreme thermophiles. Archaebacteria differs from eubacteria in ribosomal structure, the possession (in some case) of introns, and other features including membrane composition.

As used herein, “virus” refers to obligate intracellular parasites of living but non-cellular nature, consisting of DNA or RNA and a protein coat. Viruses range in diameter from about 20 to about 300 nm. Class I viruses (Baltimore classification) have a double-stranded DNA as their genome; Class II viruses have a single-stranded DNA as their genome; Class III viruses have a double-stranded RNA as their genome; Class IV viruses have a positive single-stranded RNA as their genome, the genome itself acting as mRNA; Class V viruses have a negative single-stranded RNA as their genome used as a template for mRNA synthesis; and Class VI viruses have a positive single-stranded RNA genome but with a DNA intermediate not only in replication but also in mRNA synthesis. The majority of viruses are recognized by the diseases they cause in plants, animals and prokaryotes. Viruses of prokaryotes are known as bacteriophages.

As used herein, “fungi” refers to a division of eucaryotic organisms that grow in irregular masses, without roots, stems, or leaves, and are devoid of chlorophyll or other pigments capable of photosynthesis. Each organism (thallus) is unicellular to filamentous, and possess branched somatic structures (hyphae) surrounded by cell walls containing glucan or chitin or both, and containing true nuclei.

As used herein, “an effective amount of a compound for treating a particular disease” is an amount that is sufficient to ameliorate, or in some manner reduce the symptoms associated with the disease. Such amount may be administered as a single dosage or may be administered according to a regimen, whereby it is effective. The amount may cure the disease but, typically, is administered in order to ameliorate the symptoms of the disease. Repeated administration may be required to achieve the desired amelioration of symptoms.

As used herein, “treatment” means any manner in which the symptoms of a condition, disorder or disease are ameliorated or otherwise beneficially altered. Treatment also encompasses any pharmaceutical use of the compositions herein.

As used herein, “amelioration” of the symptoms of a particular disorder by administration of a particular pharmaceutical composition refers to any lessening, whether permanent or temporary, lasting or transient that can be attributed to or associated with administration of the composition.

As used herein, “production by recombinant means” refers to production methods that use recombinant nucleic acid methods that rely on well known methods of molecular biology for expressing proteins encoded by cloned nucleic acids.

As used herein, “pharmaceutically acceptable salts, esters or other derivatives” include any salts, esters or derivatives that may be readily prepared by those of skill in this art using known methods for such derivatization and that produce compounds that may be administered to animals or humans without substantial toxic effects and that either are pharmaceutically active or are prodrugs.

As used herein, a “prodrug” is a compound that, upon in vivo administration, is metabolized or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

### B. EXEMPLARY EMBODIMENTS

Hepatitis delta virus (HDV) infections cause both acute and chronic liver disease and can be fatal (1, 2). This RNA virus contains a 1.7 kb single-stranded circular genome and delta antigen, the only known HDV-encoded protein. These elements are encapsulated by a lipid envelope in which hepatitis B virus surface antigens are embedded (3), which explains why HDV infections occur only in the presence of an accompanying HBV infection (4, 5). Two isoforms of delta antigen exist in infected livers and serum (6, 7). This heterogeneity arises from a unidirectional mutation at a single nucleotide in the termination codon for delta antigen (codon 196:UAG.fwdarw.UGG), which occurs during replication (8). Thus, although small delta antigen is 195 amino acids long, large delta antigen is identical in sequence except that it contains an additional 19 amino acids at its COOH terminus. Although both forms of delta antigen contain the same RNA genome binding domain (9), they have dramatically different effects on genome replication. The small form is required for replication, whereas the large form is a potent trans-dominant inhibitor (10, 11).

The last four amino acids of large delta antigen are Cys-Arg-Pro-Gln-COOH (SEQ ID NO:2). This COOH-terminal configuration, termed a CXXX box (where C is cysteine and X is any amino acid), has been implicated as a substrate for prenyltransferases that add to the cysteine 15 (farnesyl) or 20 (geranylgeranyl) carbon moieties derived from mevalonic acid (12-14). The resulting hydrophobic modification may aid in membrane association of the derivatized protein, as suggested for p21 Ras (15, 16) and lamin B (12, 17). We have now demonstrated that large delta antigen is similarly modified.

Other virions also contain suitable target sequences for prenylation. These sequences are near the carboxy terminus of the viral protein targeted, and may be in the form of CXXX boxes, but the cysteine may also be closer to the C-terminus, including a position as the C-terminal amino acid, as is the case of the core antigen of hepatitis B virus (HBV) and the nef gene product of HIV-1.

To determine whether large delta antigen is a substrate for prenylation, we labeled three cell lines, SAG, LAG, and GP4F, with [³H]mevalonic acid. GP4F cells are a derivative of NIH 3T3 cells (18). SAG (19) and LAG (20) cells are derivatives of GP4F cells that stably express the small and large delta antigens, respectively.

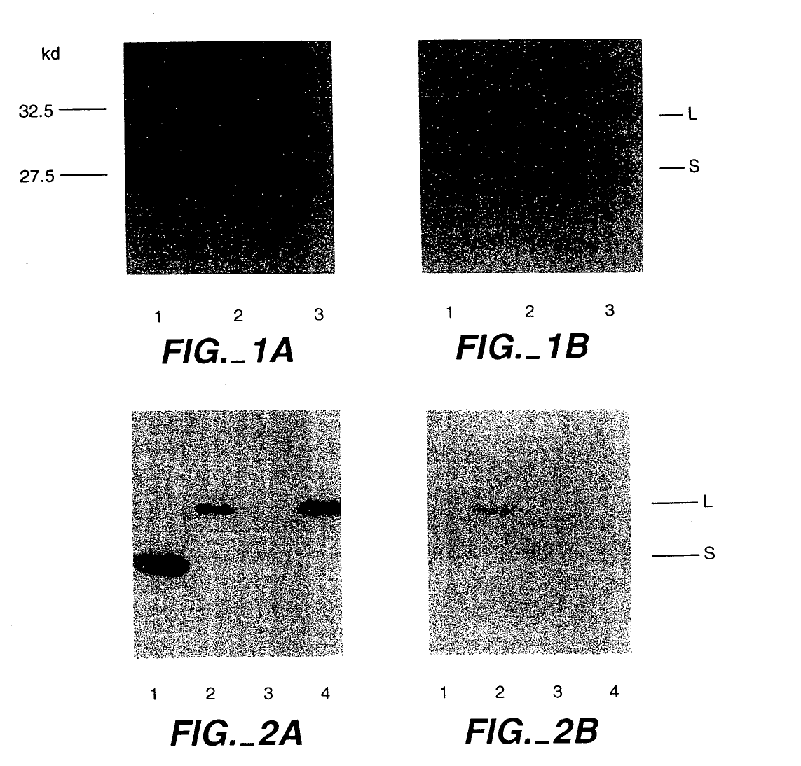

Labeled cell lysates were analyzed on immunoblots (FIG. 1A) to detect steady-state amounts of small and large delta antigen. The lysates were also subjected to immunoprecipitation with an antibody to the delta antigens (anti-delta), SDS polyacrylamide gel electrophoresis (SDS-PAGE), and fluorography (FIG. 1B).

In more detail, referring to FIG. 1, large delta antigen is shown to be prenylated in cultured cells. The cell lines SAG (19) (lane 1), LAG (20) (lane 2), and GP4F (18) (lane 3) were grown overnight in Lovastatin (25 μM) and (R,S)-[5-³H]mevalonate (140 mM)) (28), and lysed in RIPA buffer [50 mM Tris (pH 7.5), 150 mM NaCl, 1% NP-40, 0.5% sodium deoxycholate, 0.1% SDS) (20). (A) Aliquots were subjected to immunoblot analysis (11). The blot was treated with serum from an HDV-infected patient that contained antibody to delta antigen (α-βAg) and horseradish peroxidase-conjugated rabbit antibody to human immunoglobulin G (IgG) (Promega), followed by chemiluminescence (Amersham) development. (B) Immunoprecipitates (with α-βAg) from cell extracts were subjected to SDS-PAGE and fluorography. As shown in FIG. 1, S, small delta antigen, L, large delta antigen. Molecular size markers are shown at the left (in kilodaltons).

Thus, the large, but not the small, antigen was labeled with [³H]-mevalonic acid, suggesting that large delta antigen undergoes prenylation in cultured cells.

We obtained similar results using in vitro translation reactions (13) performed in the presence of [³H]proline or [³H]mevalonate (FIG. 2). FIG. 2. also shows mutation of Cys₂₁₁ of large delta antigen to Ser and loss of prenylation. In vitro translation reactions were performed with rabbit reticulocyte lysates (Promega) in the presence of either (A) L-[2,3,4,5-³H]proline (19 μM) (94 Ci/mmol, Amersham) or (B), [³H]mevalonate (200 μM) (30). For (A) and (B), translation reactions contained small delta antigen mRNA (lane 1); large delta antigen mRNA (lane 2); water (lane 3); or large delta antigen (Cys₂₁₁→Ser) (20) mRNA (lane 4). A portion (20 μl) of each reaction was added to 1 ml of RIPA buffer, immunoprecipitated with α-βAg, and analyzed as described (FIG. 1).

Both the small and the large antigens were labeled with [³H]proline (FIG. 2A), whereas only the large isoform was labeled with [³H]mevalonate (FIG. 2B). To determine whether modification by [³H]mevalonate was dependent on the presence of Cys₂₁₁ in the terminal CXXX box, we constructed a mutant that contains a serine at this position (20). Cys₂₁₁ is the only cysteine in large delta antigen. Mutating Cys₂₁₁ to Ser did not interfere with the synthesis of large delta antigen (FIG. 2A) but abolished its modification by [³H]mevalonate (FIG. 2B).

Although the first described CXXX boxes contained aliphatic residues at the first and second positions after Cys, other types of amino acids can be found in prenylation sites (13, 14).

For HDV particle formation, delta antigen and associated genomes are presumably targeted to cell membranes that contain HBV envelope proteins. We hypothesized that prenylation of large delta antigen could be involved in this process. We first examined whether large delta antigen was sufficient for HDV-like particle formation. HBV surface antigen (HBsAg) was expressed transiently in COS-7 cells together with small or large delta antigen. Virus-like particles consisting of delta antigen packaged into HBsAg-containing envelopes were analyzed by immunoprecipitation of clarified media supernatants with an antibody to HBsAg (anti-HBs).

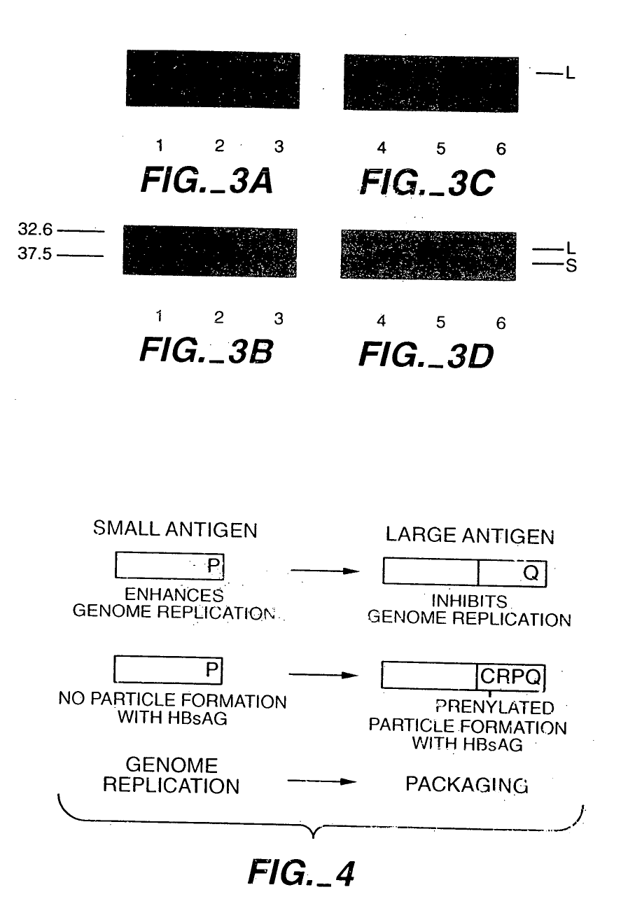

FIG. 3 shows particle formation with large delta antigen and HBsAg parts. For panels (A) and (B), COS-7 cells were transiently transfected with the following plasmids: SV24H, which expresses HBV surface antigen (29), and SVLAg, which expresses small delta antigen (19) (lane 1); SV24H and SVL-large, which expresses large delta antigen (20) (lane 2); and calcium phosphate precipitate without DNA (lane 3). In (C) and (D), COS-7 cells were transfected with SV24H and SVL-large (lane 4); SV24H and SVL-large (Ser 21) (20) (lane 5); and calcium phosphate precipitate without DNA (lane 6). For (A) and (C), 48 hours after transfection, HBsAg-containing particles were immunoprecipitated from 2-ml aliquots of clarified media supernatants with anti-HBs (29) and subjected to immunoblot (with α-βAg) and chemiluminescence analyses as described (FIG. 1). For (B) and (D), the transfected cells were harvested in cell lysis buffer [50 mM Tris (pH 8.8), 2% SDS] with protease inhibitors (20), and aliquots subjected to protein immunoblot and chemiluminescence analyses. Molecular size markers are shown at the left (in kD).

The presence of delta antigen in the immunoprecipitates was assayed by immunoblot analysis (FIG. 3A). Although both small and large antigens were synthesized in the transfected cells (FIG. 3B), only the large isoform was incorporated into secreted HBsAg-containing particles (FIG. 3A). Similar selective packaging has been observed (21).

We then examined the function of mevalonate modification in this particle formation. One explanation for the preferred packaging of large delta antigen is that the small antigen lacks the CXXX box and therefore cannot undergo modification. The Cys₂₁₁ Ser mutant of large delta antigen should behave like small delta antigen and not be packaged. This was indeed found to be the case. Whereas both wild-type and Ser 211 mutant large antigens were synthesized in transfected cells (FIG. 3D), only the wild-type form was packaged into particles (FIG. 3C). Thus, the mutated form of large delta antigen is not prenylated and cannot form particles with HBsAg.

Our results suggest that prenylation of large delta antigen is required for the formation and release of particles containing delta antigen and HBV surface antigens. The requirement of a prenylation site for productive viral infection is further suggested by the conservation of Cys 211 and a CXXX box motif among all sequenced HDV isolates (22).

The ability of large, but not small, delta antigen to be prenylated and packaged into virus particles further highlights the significance of the mutation-induced heterogeneity at the termination codon of the small delta antigen. During HDV replication, S genomes (encoding the small antigen) mutate to L genomes (encoding the large antigen). At least two effects attributable to this mutation can be distinguished (see FIG. 4). FIG. 4 shows the regulatory switch of S genomes to L genomes. During replication, S genomes encoding the small delta antigen mutate to L genomes, which encode the large delta antigen. This single base mutation has two effects on the COOH-terminus of delta antigen. The first is to change the nature of the COOH-terminal amino acid; Pro (P), which enhances genome replication (20), is replaced by Gln (Q), resulting in inhibition of genome replication. The second effect is the creation of a target prenylation site (CRPQ), C, cysteine; R, arginine; P, proline; Q, glutamine.

Thus, the first effect is the conversion of an enhancer of genome replication (small delta antigen) into a potent trans-dominant inhibitor (large delta antigen) (10, 11). This dramatic difference in function appears to be determined solely by the nature of the COOH-terminal amino acid with proline being sufficient to confer enhancer activity (11, 23). The second effect is the addition of a CXXX box to delta antigen, which allows the protein to be prenylated and presumably promotes its incorporation into HBsAg-containing particles. The combined effects of the switch from production of small to large delta antigen thus appear to have two roles: to suppress further genome replication and to promote the onset of packaging and virion morphogenesis.

Our results suggest prenylation as a new target for anti-HDV therapy and for antiviral therapy with respect to other viruses with prenylated proteins. Such therapy is directed at inhibiting virion morphogenesis, production, release and uncoating (functionally the reverse reaction of virion morphogenesis). In light of the increasingly apparent degeneracy of the four C-terminal amino acids required to function as a prenylation substrate, a cysteine located at any of these C-terminal positions is also considered to identify a potential target of antiprenylation therapy.

Several strategies designed to interfere with the prenylation stage of the HDV life cycle may be considered, including drugs that inhibit enzymes along the prenylation pathway, and CXXX box analogs. Both therapies have been considered for the inhibition of ras-mediated oncogenic transformation (24). Tetrapeptides that correspond to the CXXX box of p21 Ha-Ras inhibit prenylation of p21 Ha-Ras in vitro (25). Finally, the dual function of large delta antigen in the HDV life cycle suggests a further refinement of a proposed (11) defective interfering particle-(DIP) (26) like therapy aimed at cells infected with actively replicating S genomes. Because L genomes require a source of small delta antigen for replication (19, 27) but, once replicated, produce a potent trans-dominant inhibitor of further replication, a therapeutically administered L genome DIP could be specific for infected cells, as well as possess an inherent shut-off mechanism (11). If the L genome also contained the Cys₂₁₁→Ser mutation, it could encode a delta antigen that not only inhibits replication but also affects packaging.

Accordingly, new approaches to antiviral therapy and inhibition of viral morphogenesis focus on inhibition of the prenylation of, or post-prenylation reactions of, at least one viral protein. This may be effected by contacting cells infected with the target virus with an effective amount of an agent which inhibits the prenylation of, or post-prenylation reactions of, at least one viral protein. Such agents include inhibitors of formation of the prenyl groups which are derivative of the mevalonate synthesis pathway. Other agents include decoys for the target sequence for prenylation, including small peptides, including tetrapeptides and other compounds which mimic the surroundings of the cysteine residue to be prenylated. For example, Reiss, Y., et al. Cell (1990) 62:81-88 report prenylation inhibition by C-A-A-X (SEQ ID NO:7) tetrapeptides. As set forth above, the cysteine residue to be prenylated is generally found at the carboxy terminus of the target protein; although the most common target sequence involves a CXXX box, cysteines positioned closer to the C-terminus may also be targeted; thus, the relevant peptides may include those of the form XCXX, XXCX, and XXXC. Other agents include derivatives and mimics of prenyl groups themselves. Other suitable agents include inhibitors of the prenyltransferase enzymes and of enzymes that catalyze post-prenylation reactions.

Assay of Candidate Inhibitors

The present invention also provides a method to screen candidate drugs as prenylation inhibitors by taking advantage of the requirement for prenylation in order to effect secretion of certain prenylated proteins. For those proteins for which secretion requires prenylation, the assay can be conducted in a direct and simple manner. Cells that secrete, or that have been modified to secrete, a first protein whose secretion is dependent on prenylation are used as the experimental cells. A second protein which does not depend on prenylation for secretion is used as a control. This control protein may be secreted by the same or different host cells as the first protein. The candidate drug is applied to cells that secrete both proteins, or to matched sets of cells that secrete each. Secretion can readily be assessed by assaying the cell supernatants for the presence or absence of the first and second secreted proteins using, for example, routine ELISA assays. Successful candidate drugs will not inhibit the secretion of the control protein, but will inhibit the secretion of the protein in the test sample wherein prenylation is required for secretion.

The large delta antigen of HDV is a viral protein for which prenylation is a prerequisite for secretion. Thus, this protein forms, itself a key part of a useful test system for the assay. Cells that are modified to secrete a protein for which prenylation is not required can be used as controls. If large delta antigen is used as the test protein, it is advantageous to use HBsAg as the control protein in the same cell since HBsAg is also required for secretion of delta antigen.

The foregoing assay, of course, requires that the inhibitor interfere with the prenylation system for large delta antigen or for any other prenylation-controlled secreted protein used in the assay. A range of prenyl transferases and prenyl groups is known to apply to various proteins for which prenylation inhibitors are required or sought. Some of these proteins are not secreted, whether they are prenylated or not; one such example is the protein product of the ras oncogene.

Nevertheless, the assay system described can be employed to screen for inhibitors of prenylation in these nonsecreted proteins by providing the target “CXXX” box characteristic of the nonsecreted protein in place of the corresponding “CXXX” box of the secreted one. The resulting chimeric protein will exhibit the prenylation characteristics of the imported “CXXX” box characteristic of the nonsecreted protein, but retain the ability of the host secreted protein to be passed to the supernatant for assay. Thus, the range of target proteins for which prenylation inhibitors are sought by use of the assay can be expanded to nonsecreted proteins.

The presence of a control system which provides secreted protein not dependent on prenylation is critical. The presence of this control allows candidate inhibitors which merely are toxic to the cells, or which inhibit secretion in general, to be discarded. Prenylation inhibitors identified by one of the variations of the above described assay are expected to find use not only in the inhibition of viruses, but also in other processes or disease states—including but not limited to cancer—in which a prenylated protein is found to be involved.

Evidently, prenylation of viral proteins is a prerequisite for additional post-prenylation reactions of the proteins such as proteolysis and carboxymethylation. The essential sequence of steps can be interfered with at the most convenient point for the viral protein in question.

Administration of the Inhibitors

Additional viral proteins subject to prenylation can be obtained by screening amino acid sequence data banks for viral proteins which contain a “CXXX” box at the C-terminus or other Cys-contained sequences near the C-terminus. An illustrative list of such proteins includes, for example, specific proteins of HAV, HCV, HSV, CMV, VZV, influenza virus, plant viruses such as tobacco mosaic satellite virus and barley stripe mosaic virus, core antigen of hepatitis B virus and the nef gene product of HIV I, as set forth above. These candidates for suitable prenylation targets can be validated in a manner similar to that described above by providing labeled mevalonic acid to cells infected with or containing the appropriate viruses or viral gene products, and assessing the prenylation status of the viral proteins obtained using incorporation of label as the criterion. Furthermore, the role of prenylation in the morphogenesis of the respective virions, and its suitability as a target for anti-viral therapy, can also be validated in a manner similar to that described above.

If viral morphogenesis, production, release or uncoating are to be inhibited in culture, suitable host cells are used to culture the virus, and the agents used in inhibiting prenylation or post prenylation reactions added to the medium. If the infected cells are contained in an animal subject, such as a mammalian subject or in particular a human or other primate subject, the agent used for the prenylation inhibition is generally introduced as a pharmaceutical formulation. Suitable formulations depending on the nature of the agent chosen may be found in Remington's Pharmaceutical Sciences, latest edition, Mack Publishing Co., Easton, Pa. The routes of administration include standard such routes, including administration by injection, oral administration, and transmucosal and transdermal administration. The choice of formulation will depend on the route of administration as well as the agent chosen. Suitable mixtures of agents can also be used as active ingredients. For administration to plants, formulations which are capable of conducting the active ingredients into plant cells are used as carriers.

### C. THE FORMULATION, DOSAGE AND ROUTE OF ADMINISTRATION OF ANTIVIRAL AGENT

The formulation, dosage and route of administration of the above-described antiviral agents, preferably in the form of pharmaceutical compositions, can be determined according to the methods known in the art (see e.g., Remington: The Science and Practice of Pharmacy, Alfonso R. Gennaro (Editor) Mack Publishing Company, April 1997; Therapeutic Peptides and Proteins: Fonnulation, Processing, and Delivery Systems, Banga, 1999; and Pharmaceutical Formulation Development of Peptides and Proteins, Hovgaard and Frkjr (Ed.), Taylor & Francis, Inc., 2000; Medical Applications of Liposomes, Lasic and Papahadjopoulos (Ed.), Elsevier Science, 1998; Textbook of Gene Therapy, Jain, Hogrefe & Huber Publishers, 1998; Adenoviruses: Basic Biology to Gene Therapy, Vol. 15, Seth, Landes Bioscience, 1999; Biopharmaceutical Drug Design and Development, Wu-Pong and Rojanasakul (Ed.), Humana Press, 1999; Therapeutic Angiogenesis: From Basic Science to the Clinic, Vol. 28, Dole et al. (Ed.), Springer-Verlag New York, 1999). The antiviral agent can be formulated for oral, rectal, topical, inhalational, buccal (e.g., sublingual), parenteral (e.g., subcutaneous, intramuscular, intradermal, or intravenous), transdermal administration or any other suitable route of administration. The most suitable route in any given case will depend on the nature and severity of the condition being treated and on the nature of the particular antiviral agent that is being used.

The antiviral agent can be administered alone. Alternatively and preferably, the antiviral agent is co-administered with a pharmaceutically acceptable carrier or excipient. Any suitable pharmaceutically acceptable carrier or excipient can be used in the present method (See e.g., Remington: The Science and Practice of Pharmacy, Alfonso R. Gennaro (Editor) Mack Publishing Company, April 1997).

The present method can be used alone. Alternatively, the present method can be used in combination with other agent suitable for treating a viral infection. Such other agent can be used before, with or after the administration of the above-described antiviral agent.

According to the present invention, the antiviral agent, alone or in combination with other agents, carriers or excipients, may be formulated for any suitable administration route, such as intracavernous injection, subcutaneous injection, intravenous injection, intramuscular injection, intradermal injection, oral or topical administration. The method may employ formulations for injectable administration in unit dosage form, in ampoules or in multidose containers, with an added preservative. The formulations may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, sterile pyrogen-free water or other solvents, before use. Topical administration in the present invention may employ the use of a foam, gel, cream, ointment, transdermal patch, or paste.

Pharmaceutically acceptable compositions and methods for their administration that may be employed for use in this invention include, but are not limited to those described in U.S. Pat. Nos. 5,736,154; 6,197,801 B1; 5,741,511; 5,886,039; 5,941,868; 6,258,374 B1; and 5,686,102.

The magnitude of a therapeutic dose in the treatment or prevention will vary with the severity of the condition to be treated and the route of administration. The dose, and perhaps dose frequency, will also vary according to age, body weight, condition and response of the individual patient.

It should be noted that the attending physician would know how to and when to terminate, interrupt or adjust therapy to lower dosage due to toxicity, or adverse effects. Conversely, the physician would also know how to and when to adjust treatment to higher levels if the clinical response is not adequate (precluding toxic side effects).

Any suitable route of administration may be used. Dosage forms include tablets, troches, cachet, dispersions, suspensions, solutions, capsules, patches, and the like. See, Remington's Pharmaceutical Sciences.

In practical use, the antiviral agent, alone or in combination with other agents, may be combined as the active in intimate admixture with a pharmaceutical carrier or excipient, such as beta-cyclodextrin and 2-hydroxy-propyl-beta-cyclodextrin, according to conventional pharmaceutical compounding techniques. The carrier may take a wide form of preparation desired for administration, topical or parenteral. In preparing compositions for parenteral dosage form, such as intravenous injection or infusion, similar pharmaceutical media may be employed, water, glycols, oils, buffers, sugar, preservatives, liposomes, and the like known to those of skill in the art. Examples of such parenteral compositions include, but are not limited to dextrose 5% w/v, normal saline or other solutions. The total dose of the antiviral agent, alone or in combination with other agents to be administered may be administered in a vial of intravenous fluid, ranging from about 1 ml to 2000 ml. The volume of dilution fluid will vary according to the total dose administered.

The invention also provides for kits for carrying out the therapeutic regimens of the invention. Such kits comprise in one or more containers therapeutically effective amounts of the antiviral agent, alone or in combination with other agents, in pharmaceutically acceptable form. Preferred pharmaceutical forms would be in combination with sterile saline, dextrose solution, or buffered solution, or other pharmaceutically acceptable sterile fluid. Alternatively, the composition may be lyophilized or dessicated; in this instance, the kit optionally further comprises in a container a pharmaceutically acceptable solution, preferably sterile, to reconstitute the complex to form a solution for injection purposes. Exemplary pharmaceutically acceptable solutions are saline and dextrose solution.

In another embodiment, a kit of the invention further comprises a needle or syringe, preferably packaged in sterile form, for injecting the composition, and/or a packaged alcohol pad. Instructions are optionally included for administration of composition by a physician or by the patient.

### D. EXAMPLES

The following experiments demonstrate that inhibition of prenylation of viral proteins in vivo and/or in vitro can inhibit or retard reproduction of three representative viruses: hepatitis delta virus (HDV), a negative single-stranded RNA virus; vaccinia virus, a double-stranded DNA virus; and Hepatitis A virus (HAV), a positive single-stranded RNA virus.

### Example 1

### Prenylation of the Viral CXXX Boxes

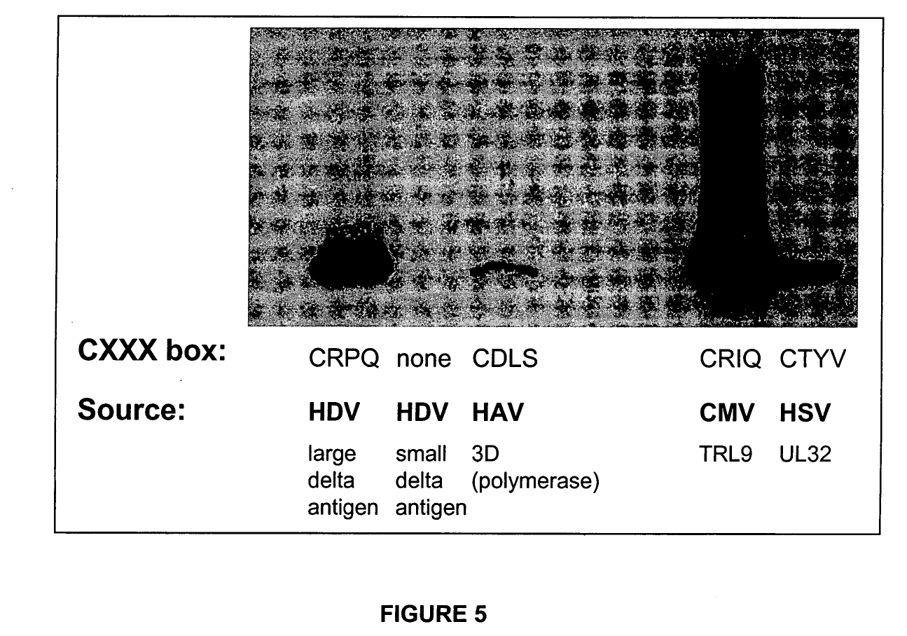

Experiment were conducted to demonstrate that CXXX boxes of several distinct viral proteins can serve as substrates for prenylation and their prenylation can be inhibited by prenylation inhibitors (FIG. 5).

CXXX boxes from the proteins of several representative viruses were selected:

1) -CDLS (SEQ ID NO:8) is the CXXX box from the 3D protein (replication polymerase protein) of hepatitis A virus (HAV). The virus is known to replicate in association with cellular membranes. Therefore, prenylation of the replication protein is an ideal antiviral target;

2) -CTYV (SEQ ID NO:9) is the CXXX box from the UL32 protein of herpes simplex virus (HSV). There is genetic evidence showing that the UL32 protein is important for production of HSV virus particles. It is not required for replication of the viral genome, but rather appears to act in assembly of the virus. Prenylation of UL32 would be an ideal mechanism to anchor nascent particle assembly to the intracellular membrane sites where assembly occurs; and

3) -CRIQ (SEQ ID NO:10) is the CXXX box of TRL9 from cytomegalovirus (CMV). TRL9 was chosen as an example of an open reading frame encoded in a virus, but of completely unknown function. As such, there is no inherent bias in its selection attributable to any inferred role in its encoding virus' lifecycle. It can thus serve as a “generic” viral CXXX box.

The above-selected CXXX boxes are representative because HDV has a circular, negative-stranded RNA genome, HAV has a linear, plus-stranded RNA genome, and HSV and CMV have double-stranded DNA genomes.

These CXXX boxes were substituted for the native CXXX box of the large delta antigen using PCR mutagenesis and standard molecular cloning techniques. The following oligo pairs were used for the PCR mutagenesis:

1) For CDLS Construct (Hepatitis A Virus):

| | |
|---|---|
| 5′-GGCTTCGTCCCCAGTCTGCAGGGAGTCCCGG-3′ | (SEQ ID NO:11) |
| and | |
| 5′-GGGGCCGGATCCCGCTTTATTTACGAGAGGTCACAACTCTGGGG-3′. | (SEQ ID NO:12) |

2) For CTYV Construct (Herpes Simplex Virus):

| | |
|---|---|
| 5′-GGCTTCGTCCCCAGTCTGCAGGGAGTCCCGG-3′ | (SEQ ID NO:13) |
| and | |
| 5′-GGGGCCGGATCCCGCTTTATTTACACGTATGTACAACTCTGGGG-3′. | (SEQ ID NO:14) |

3) For CRIQ Construct (Cytomegalovirus):

| | |
|---|---|
| 5′-GGCTTCGTCCCCAGTCTGCAGGGAGTCCCGG-3′ | (SEQ ID NO:15) |
| and | |
| 5′-GGGGCCGGATCCCGCTTTATTTACTGGATTCGACAACTCTGGGG-3′. | (SEQ ID NO:16) |

The chimeric proteins were expressed in rabbit reticulocyte lysates, which are known to contain active prenyltransferases, in the presence of ³H-mevalonate (the metabolic precursor of prenyl lipids), using the procedures described in Glenn et al, Science, 256:1331-1333 (1992). Also expressed in parallel were large delta antigen with its native CXXX box, as positive control, and small delta antigen which lacks a CXXX box, as negative control.

The expressed proteins were isolated by immunoprecipitation with an antibody specific for delta antigen, and analyzed by SDS-polyacrylamide gel electrophoresis followed by transfer to nitrocellulose membrane and fluorography, as described in Glenn et al, Science, 256:1331-1333 (1992).

The results show that the viral CXXX boxes from the three chimeric proteins can all be prenylated (FIG. 1). As expected, the CXXX box from the native large delta antigen was also prenylated, and the small delta antigen which lacks a CXXX box and should therefore not be a substrate for prenylation indeed remained completely unmodified.

It is thus shown that CXXX boxes from different types of proteins ranging from well characterized to still unknown functions encoded in several quite distinct classes of viruses can be prenylated.

### Example 2

### Inhibition of HDV Virion Production

In vitro inhibition

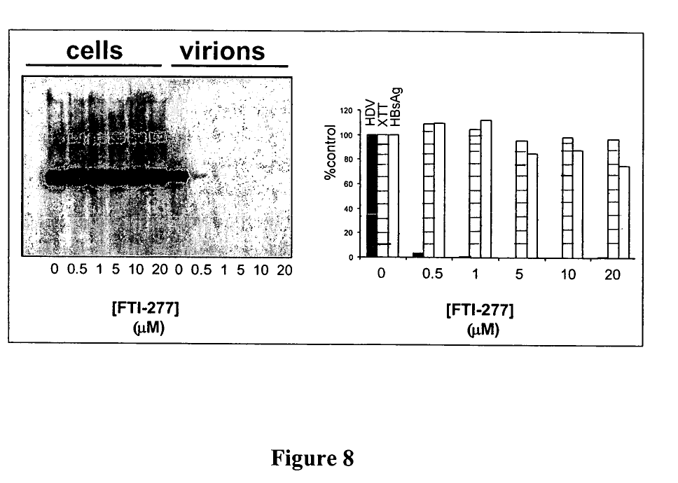

Experiments were conducted to demonstrate that FTI-277, a prenylation inhibitor, can effectively inhibit the production of HDV virions at a concentration that does not significantly affect general protein synthesis and secretion, and does not significantly affect overall cell metabolism (FIGS. 5-8).

Completely infectious HDV particles were produced using the system disclosed in Sureau et al., J. Virol., 66:1241-5 (1992). Co-transfection of Huh-7 cells, a liver-derived cell line, with plasmids encoding the complete HDV and HBV genomes yielded HDV virions released into the media supernatant (FIG. 5). Such released virions contain an intact HDV genome.

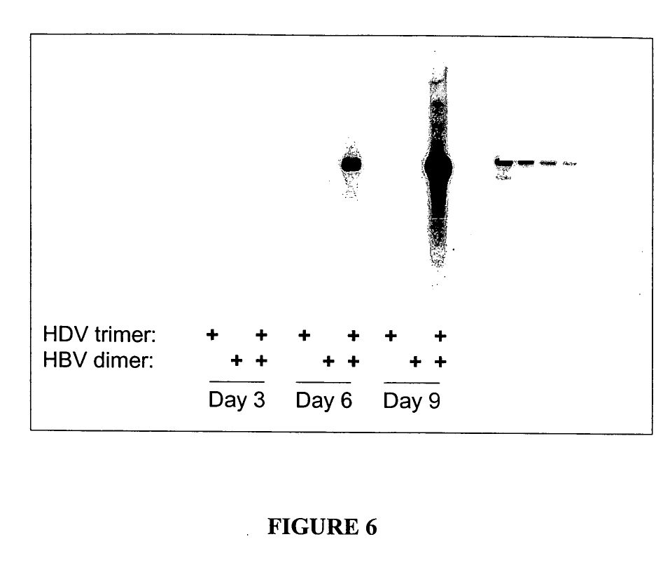

As shown in FIG. 6, one week after the produced virions were inoculated onto cultures of the human hepatocytes, at least 5-10% of the latter displayed the nuclear staining pattern characteristic of HDV infection when analyzed by immunofluoresence with an antibody against delta antigen. Thus, not only do the produced virions contain an intact RNA genome, but they are also infectious. This represents the first use of cultured human primary hepatocytes as a target for HDV infection.

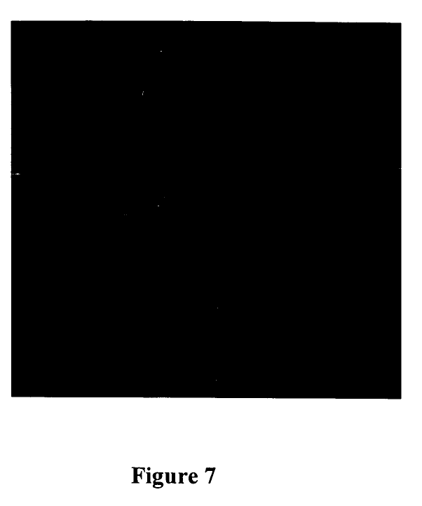

FTI-277, a prenylation inhibitor, was tested for its ability to inhibit HDV virion production. As shown in FIG. 7, while in the absence of drug virions were readily produced, they were dramatically inhibited at mid-nanomolar concentration of FTI-277. At the micromolar concentration of FTI-277, there were no detectable HDV virions produced. Non-specific toxicity was assessed by free HBV surface antigen assay, which assesses effects on general protein synthesis and secretion, and a standard XTT assay, which measures overall cell metabolism. As shown in FIG. 7, FTI-277 can effectively inhibit HDV virion production at a concentration that essentially does not affect general protein synthesis and overall cell metabolism.

Taken together, the above results demonstrate that pharmacological inhibition of prenylation can interfere with virus particle production. Furthermore, compounds like FTI-277, which inhibit prenylation, represent a novel class of antiviral agents.

In Vivo Inhibition

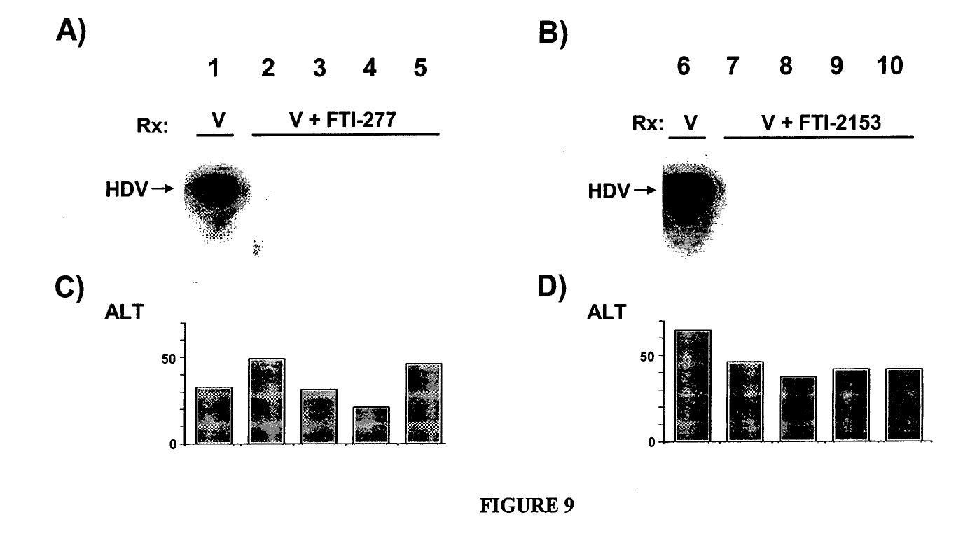

Experiments were conducted to demonstrate that: 1) the prenylation inhibitors FTI-277 and FTI-2153 can be used to treat hepatitis delta virus (HDV) infection in vivo; and 2) FTI-277 and FTI-2153 can effectively inhibit the production of HDV virions at a concentration that is not toxic to the testing animals (FIGS. 9A-D).

HBV-transgenic mice were inoculated by hydrodynamic transfection to initiate authentic HDV genome replication. Mice were treated for one week by IP injection with vehicle alone (FIGS. 9A and 9B, lanes 1 and 6), vehicle+50 mg/kg/day FTI-277 (FIGS. 9A and 9B, lanes 2-5), or vehicle+50 mg/kg/day FTI-2153 (FIGS. 9A and 9B, lanes 7-10). Serum samples were then analyzed for HDV virions by RT-PCR analysis (FIGS. 9A and 9B, lanes 1-10). The primers used in the RT-PCR assay yield a 540 bp fragment only in the presence of circular viral genomic RNA, as found in virions. The production and release of HDV virions into the serum was completely eliminated in the groups treated with prenylation inhibitors.

Non-specific toxicity of the FTI-277 and FTI-2153 on the testing animals was assessed by alanine aminotransferase (ALT) assays, which is a standard “liver function” test, (FIGS. 9C and 9D) performed on aliquots of the corresponding serum samples from FIGS. 9A and 9B. For the dosages tested, on average, animals treated with FTI-277 have the same level of ALT as the placebo and animals treated with FTI-2153 have lower level of ALT than the placebo.

Taken together, the above results demonstrate that the prenylation inhibitors FTI-277 and FTI-2153 can effectively inhibit HDV virion production in vivo. This inhibition is not associated with, and cannot be explained by, non-specific toxicity in the testing animals.

### Example 3

### Inhibition of Vaccinia Virus Production

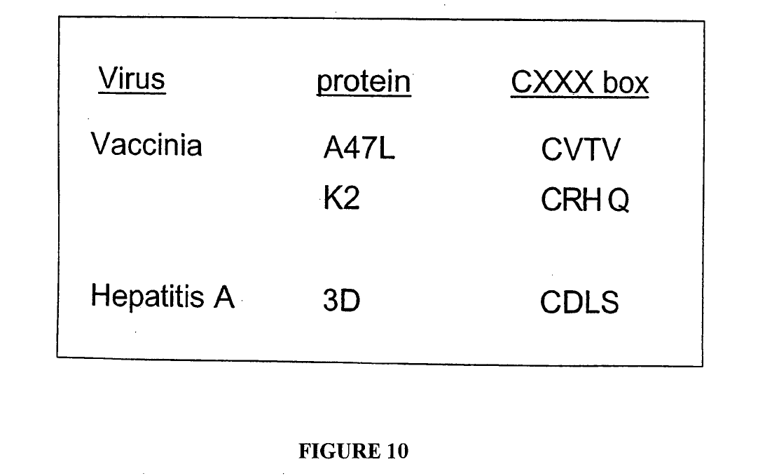

Experiments were conducted to demonstrate the dramatic effect of prenylation inhibitors on vaccinia virus production and HAV replication (FIGS. 10-12).

Vaccinia virus has several proteins with CXXX boxes, making it a claimed target for the antiviral effect of prenylation inhibitors. Hepatitis A virus (HAV) also has a protein, 3D, with a CXXX box that is prenylated (see FIG. 10). FIG. 6 shows CXXX box-containing proteins in vaccinia virus and hepatitis A virus. Proteins containing the substrate requirement for prenylation—a CXXX box—are indicated along with the specific amino acid sequence of their respective CXXX boxes. Standard single letter abbreviations are used for the CXXX box sequences.

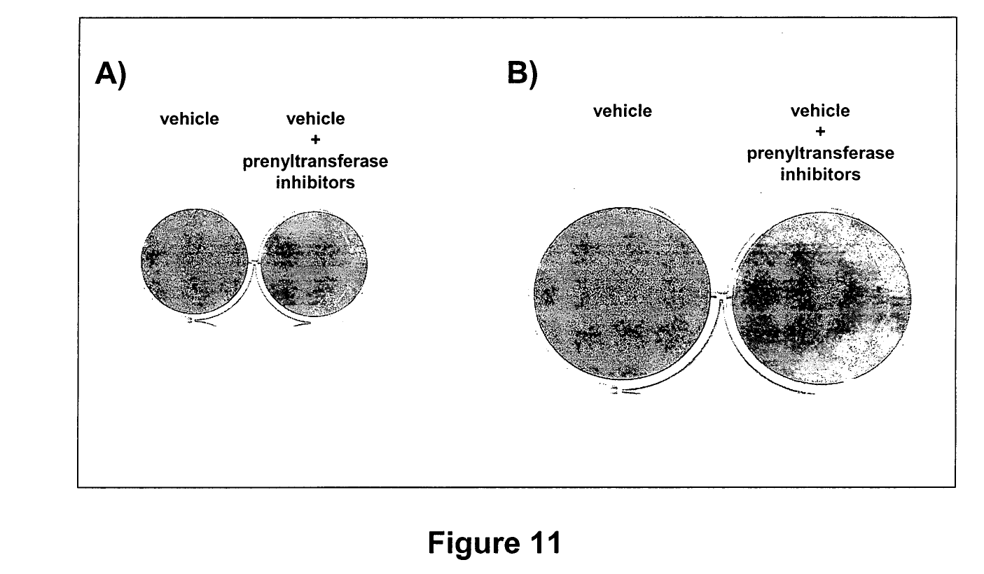

FIG. 11 demonstrates the dramatic effect of prenylation inhibitors on vaccinia virus production. A standard vaccinia virus assay was performed in which equal amounts of vaccinia virus were added to wells containing a monolayer of susceptible CV-1 cells. The wells were incubated at 37° C. in CV-1 medium containing vehicle (DMSO) alone, or vehicle plus an equimolar 10 micromolar mixture of FTI-2153 (a farnesyltransferase inhibitor) and GGTI-2166 (a geranylgeranyltransferase inhibitor) (Sun et al., Cancer Res., 59(19):4919-26 (1999)). On day 2, the cells were fixed with crystal violet to permit detection of vaccinia virus-induced plaques (i.e., holes in the CV-1 cell monolayer resulting from death of infected cells). B) is a higher power picture of A).

Although the number of plaques is similar—which is to be expected because the same amount of inoculum was added to each well—the size of the plaques obtained in the presence of prenylation inhibitors is quite obviously smaller, about 15 fold less surface area. Because plaque size reflects the ability of virus to reproduce itself and continue its life cycle by infecting new neighboring cells, this experiment shows quite nicely the antiviral effect of the prenylation inhibitors.

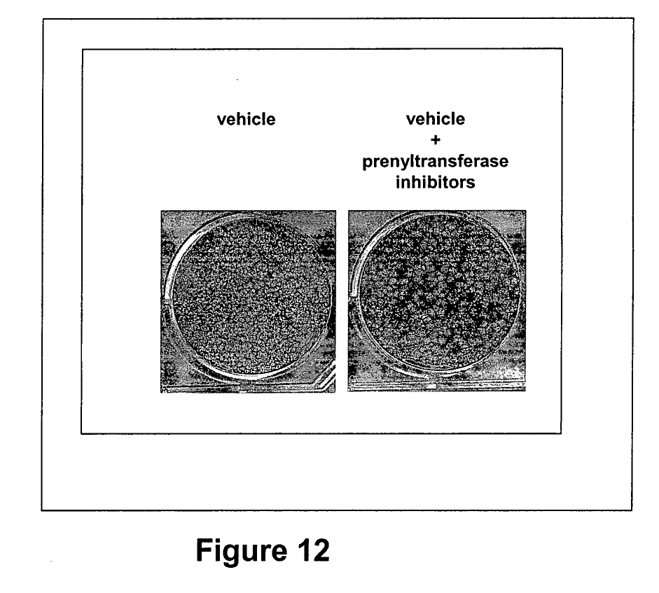

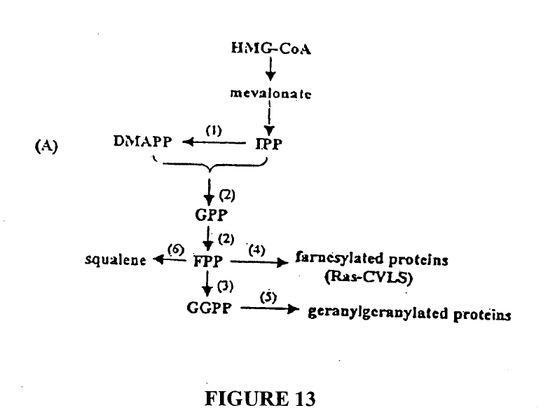

To further demonstrate that the smaller plaque sizes indeed reflect decreased total virus production, aliquots of the supernatants collected from these wells were then titered in the absence of prenylation inhibitors. FIG. 12 shows that prenylation inhibitors decrease vaccinia virus production. To measure the relative titers of virus produced in the presence and absence of prenylation inhibitors, aliquots collected from the wells in the experiment of FIG. 11 were added to fresh wells containing monolayers of CV-1 cells. The latter were incubated at 37° C. in standard CV-1 medium after which the cells were fixed with crystal violet to permit detection of vaccinia virus-induced plaques. The results are shown in FIG. 12 where each plaque obtained was derived from an individual virus particle added to the monolayer at the onset of the experiment. Indeed the total number of virus produced is approximately a log lower in the supernatants collected over cells grown in the presence of prenylation inhibitors.

### E. REFERENCES AND NOTES

The following references are listed according to the number which refers to them in the body of the specification.
1. Rizzetto, M., Hepatology (1983) 3:729.
2. Hoffnagle, J. H., J. Am. Med. Assoc. (1989) 261:1321.
3. Bonino, F., et al., Infect. Immun. (1984) 43:1000.
4. Rizzetto, M., et al., J. Infect. Dis. (1980) 141:590.
5. Rizzetto, M., et al., Proc. Natl. Acad. Sci. U.S.A. (1980) 77:6124.
6. Bergmann, K. F., et al., J. Infect. Dis. (1986) 154:702.
7. Bonino, F., et al., J. Virol (1986) 58:945.
8. Luo, G., et al., ibid. (1990) 64:1021.
9. Lin, J.-H., et al., ibid., p. 4051.
10. Chao, M., et al., ibid., p. 5066.
11. Glenn, J. S., et al., ibid. (1991) 65:2357.
12. Glomset, J. A., et al., Trends Biochem. Sci. (1990) 15:139.
13. Maltese, W. A., FASEB J. (1990) 4:3319.
14. Moores, S. L., et al., J. Biol. Chem. (1991) 266:14603.
15. Hancock, J. F., et al., Cell (1989) 57:1167.
16. Schafer, W. R., et al., Science (1989) 245:379.
17. Beck, L. A., et al., J. Cell Biol. (1988) 107:1307.
18. Ellens, H., et al., Methods Cell Biol. (1989) 31:155.
19. Glenn, J. S., et al., J. Virol. (1990) 64:3104. SAG cells are identical to GAG cells.
20. Glenn, J. S., thesis, University of California, San Francisco (1992).
21. Wang, C. J., et al., J. Virol. (1991) 65:6630; Ryu, W.-S., et al., J. Virol. (1992), 66:2310.
22. Of 14 independent viral isolates sequenced, 13 code for Cys-Arg-Pro-Gln-COOH and 1 codes for Cys-Thr-Pro-Gln-COOH as the four terminal amino acids of large delta antigen [Wang, K.-S., et al., Nature (1986) 323:508; Makino, S., et al., ibid. (1987) 329:343; Kuo, M. Y. P., et al., J. Virol. (1988) 62:1855; Saldanha, J. A. et al., J. Gen. Virol. (1990) 71:1603; Xia, Y.-P., et al., (1990) 178:331; Imazeki, F. et al., J. Virol. (1990) 64:5594; Chao, Y.-C., et al., Hepatology (1991) 13:345; Deny, P. et al., J. Gen. Virol. (1991) 72:735].
23. We have recently found that specific mutation of the COOH-terminal Gln of large delta antigen to Pro converted the protein from an inhibitor to an enhancer of genome replication (20).
24. Gibbs, J. B., Cell (1991) 65:1.
25. Reiss, Y., et al., ibid. (1990) 62:81.
26. Ramig, R. F., in Virology, Fields, B. N., et al., Eds. (Raven, N.Y., 1990), pp. 112-122.
27. Kuo, M. Y.-P., et al., J. Virol. (1989) 63:1945.
28. (R,S)-[5-³H]mevalonate (4 to 18.8 Ci/mmol) was synthesized according to the method of R. K. Keller, J. Biol. Chem. (1986) 261:12053.
29. Bruss, V. et al., J. Virol. (1991) 65:3813.

1. A method to treat a viral infection in a subject via inhibiting the prenylation or a post-prenylation reaction of a protein contained in the virus infecting said subject, which method comprises administering to said subject an effective amount of
an inhibitor of prenyl cysteine methyltransferase.

2. (canceled)

3. (canceled)

4. (canceled)

5. (canceled)

6. The method of claim 1, wherein said subject is an animal or a plant.

7. The method of claim 1, wherein said animal is a mammal.

8. The method of claim 7, wherein said mammal is a human.

9. The method of claim 7, wherein said mammal is a non-human primate.

10. The method of claim 1, wherein said viral infection is caused by a virus selected from the group consisting of a double-strand DNA virus, a negative single-strand RNA virus, a positive single-strand RNA virus and a double-strand RNA virus.

11. The method of claim 10, wherein said double-strand DNA virus is selected from the group consisting of a poxviridae, a herpesviridae and a papillomaviridiae.

12. The method of claim 10, wherein said negative single-strand RNA virus is a bunyaviridiae or a hepatitis delta virus (HDV).

13. The method of claim 10, wherein said positive single-strand RNA virus is a hepatovirus.

14. The method of claim 10, wherein said double-strand RNA virus is a reoviridiae.

15. The method of claim 1, wherein said viral infection is caused by a virus selected from the group consisting of a pox virus, a bunyavirus, hepatitis E virus, human papilloma virus, molluscum contagiosum virus, vaccinia virus and reovirus.

16. The method of claim 15, wherein said pox virus is smallpox virus.

17. The method of claim 15, wherein said bunyavirus is oropouche virus.

18. The method of claim 1, wherein said inhibitor is administered with a pharmaceutically acceptable carrier or excipient.

19. (canceled)

20. (canceled)

21. The method of claim 1, wherein said inhibitor of prenyl cysteine methyltransferase is used in combination with an effective amount of an agent selected from the group consisting of
a peptide that mimics the amino acid sequence of a “CXXX” (SEQ ID NO:1), “XCXX” (SEQ ID NO:3), “XXCX” (SEQ ID NO:4), or “XXXC” (SEQ ID NO: 5) box as it occurs in said viral protein,
an inhibitor of a prenyl transferase,
an inhibitor of an enzyme included in the pathway of a prenyl lipid synthesis from mevalonate,
a mimic of a prenyl group, and
an inhibitor of a protease that removes the XXX tripeptide from the CXXX polypeptide following prenylation, a protease that removes the XX dipeptide from the XCXX polypeptide following prenylation, or a protease that removes the X residue from the XXCX polypeptide following prenylation, or a protease that removes a C-terminal domain of the prenylated protein including the entire CXXX box.
